(19) United States
(12) Patent Application Publication
(10) Pub. No.: US 2011/0040313 A1
(43) Pub. Date: Feb. 17, 2011

(54) IMPLANTABLE RESTRICTION DEVICE WITH PROTECTIVE MEMBER

(76) Inventors: Daniel F. Dlugos, JR., Middletown, OH (US); Steven G. Hall, Cincinnati, OH (US); David T. Krumanaker, Cincinnati, OH (US); Brandon J. Lloyd, Maineville, OH (US); Amy L. Marcotte, Mason, OH (US); Jerome R. Morgan, Cincinnati, OH (US); Mark S. Ortiz, Milford, OH (US); David N. Plescia, Cincennati, OH (US); Frederick E. Shelton, IV, New Vienna, OH (US); Jeffrey S. Swayze, Hamilton, OH (US); Richard W. Timm, Cincinnati, OH (US); Lauren S. Weaner, Cincinnati, OH (US); Duane A. Granston, White Plains, NY (US)

Correspondence Address: WELSH FLAXMAN & GITLER LLC, 2000 DUKE STREET, SUITE 100, ALEXANDRIA, VA, 22314, US

(21) Appl. No.: 12/541,630

(22) Filed: Aug. 14, 2009

(51) Int. Cl. A61B 17/12

(52) U.S. Cl. 606/157

(57) ABSTRACT

An implantable restriction device includes a belt and a balloon secured to the belt for engagement with tissue when the implantable restriction device is positioned about an organ. A protective member is associated with the balloon for positioning between the balloon and a tissue surface defining a band tissue interface.

### BACKGROUND OF THE INVENTION

1. Field of the Invention

The present invention relates to an implantable restriction device. More particularly, the invention relates to the protection of both the implantable restriction device and internal tissue or lumen, such as, stomach tissue, during the application and operation of the implantable restriction device.

2. Description of the Prior Art

Morbid obesity is a serious medical condition. In fact, morbid obesity has become highly pervasive in the United States, as well as other countries, and the trend appears to be heading in a negative direction. Complications associated with morbid obesity include hypertension, diabetes, coronary artery disease, stroke, congestive heart failure, multiple orthopedic problems and pulmonary insufficiency with markedly decreased life expectancy. With this in mind, and as those skilled in the art will certainly appreciate, the monetary and physical costs associated with morbid obesity are substantial. In fact, it is estimated the costs relating to obesity are in excess of one hundred billion dollars in the United States alone.

A variety of surgical procedures have been developed to treat obesity. The most common currently performed procedure is Roux-en-Y gastric bypass (RYGB). This procedure is highly complex and is commonly utilized to treat people exhibiting morbid obesity. Other forms of bariatric surgery include Fobi pouch, bilio-pancreatic diversion, and gastroplastic or “stomach stapling”. In addition, implantable devices are known which limit the passage of food through the stomach and affect satiety.

In view of the highly invasive nature of many of these procedures, efforts have been made to develop less traumatic and less invasive procedures. Gastric-banding is one of these methods. Gastric-banding is a type of gastric reduction surgery attempting to limit food intake by reducing the size of the stomach. In contrast to RYGB and other stomach reduction procedures, gastric-banding does not require the alteration of the anatomy of the digestive tract in the duodenum or jejunum.

Since the early 1980's, gastric bands have provided an effective alternative to gastric bypass and other irreversible surgical weight loss treatments for the morbidly obese. Several alternate procedures are performed under the heading of gastric-banding. Some banding techniques employ a gastric ring, others use a band, some use stomach staples and still other procedures use a combination of rings, bands and staples. Among the procedures most commonly performed are vertical banded gastroplasty (VBG), silastic ring gastroplasty (SRG) and adjustable silastic gastric banding (AGB).

In general, the gastric band is wrapped around an upper portion of the patient's stomach, forming a stoma that is less than the normal interior diameter of the stomach. This restricts food passing from an upper portion to a lower digestive portion of the stomach. When the stoma is of an appropriate size, food held in the upper portion of the stomach provides a feeling of fullness that discourages over eating.

As those skilled in the art will certainly appreciate, hybrid procedures involving gastric bypass and the utilization of a gastric band are becoming more and more common. These hybrid procedures involve placing a gastric band about the stomach in conjunction with the performance of the surgical procedure for gastric bypass surgery. However, significant challenges are associated with this procedure and, more specifically, some of these challenges relate to the placement of the gastric band directly over a staple line created as a result of the gastric bypass surgery. When a balloon type gastric band is positioned over a staple line, potential complications are encountered. These complications include damage to the gastric band resulting from the interaction of the gastric band with the staples in a manner potentially causing puncture of the gastric band by the staples. In addition to the potential puncture of the gastric band, the interaction of the gastric band with the staples may cause damage to the staple line due to rubbing of the staple line by positioning of the gastric band thereover and infection at the site of the gastric band placement.

### SUMMARY OF THE INVENTION

It is, therefore, an object of the present invention to provide an implantable restriction device including a belt and a balloon secured to the belt for engagement with tissue when the implantable restriction device is positioned about an organ. A protective member is associated with the balloon for positioning between the balloon and a tissue surface defining a band tissue interface.

It is a further object of the present invention to provide an implantable restriction device wherein the protective member includes a protective plate which is flexible and positioned along the inner surface of the balloon of the restrictive device.

It is also an object of the present invention to provide an implantable restriction device wherein the protective member includes a protective plate which is pleated and positioned along the inner surface of the balloon of the restrictive device.

It is another object of the present invention to provide an implantable restriction device wherein the protective plate is in the form of a directional flexible plate.

It is a further object of the present invention to provide an implantable restriction device wherein the protective plate is made of expandable PTFE.

It is also an object of the present invention to provide an implantable restriction device wherein the protective member is an overlapping plate system and the overlapping plate system utilizes an intralocking and overlapping construction that can accordion open or close to protect the balloon.

It is another object of the present invention to provide an implantable restriction device wherein the protective member is a sponge that conforms to the band tissue interface so as to prevent undesirable interaction which might ultimately cause damage to either the staple line or the balloon.

It is a further object of the present invention to provide an implantable restriction device wherein the protective member is an expandable PTFE applied to the inner surface of the balloon of the implantable restriction device.

It is also an object of the present invention to provide an implantable restriction device wherein the protective member is composed of stand-offs formed along the inner surface of the balloon.

It is another object of the present invention to provide an implantable restriction device wherein the stand-offs are of a different durometer than the balloon.

It is a further object of the present invention to provide an implantable restriction device wherein the stand-offs are shaped and dimensioned to straddle the staple line and protect the balloon.

It is also an object of the present invention to provide an implantable restriction device wherein the protective member is a protective layer added to the balloon that interfaces with the staple line and wherein a section of the balloon in contact with the staple line is formed from a material having a different durometer than the remainder of the balloon.

It is another object of the present invention to provide an implantable restriction device wherein the protective member is a fibrous mesh within an inner wall of the balloon.

It is a further object of the present invention to provide an implantable restriction device wherein the protective member is a self sealing material.

It is also an object of the present invention to provide an implantable restriction device wherein the self sealing material is an inner wall of the balloon.

It is another object of the present invention to provide an implantable restriction device wherein the protective member is integrally associated with the balloon.

It is a further object of the present invention to provide an implantable restriction device wherein the protective member includes a plurality of individual lumens in a layered configuration.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view showing an embodiment of a gastric band in accordance with the present invention applied about the stomach.

FIG. 2 is a perspective view of the gastric band shown in FIG. 1.

FIGS. 3-7 are top views of gastric bands applied about a sleeve portion of the stomach in accordance with various embodiments of the present invention.

FIGS. 8, 9 and 10 are perspective views of gastric bands in accordance with various alternate embodiments of the present invention.

FIGS. 11 and 12 are cross sectional views showing balloons constructed in accordance with alternate embodiments of the present invention.

FIG. 13 is a top view of a gastric band applied about a sleeve portion of the stomach in accordance with another embodiment of the present invention.

FIGS. 14-20 show various embodiments of a gastric band employing a balloon with a self sealing inner surface.

FIGS. 21, 22 and 23 are various views showing the construction of a layered balloon employed in a gastric band in accordance with the present invention.

FIGS. 24, 25, and 26 are respectively a top view about a sleeve portion of the stomach, a cross sectional view and a cross sectional view showing embodiments of a gastric band employing cellular structure.

FIG. 27 is a cross sectional view of a balloon employed in an alternate embodiment of a gastric band in accordance with the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and for teaching one skilled in the art how to make and/or use the invention.

As discussed above, the present invention relates to an implantable restriction device. A preferred embodiment of the implantable restriction device is disclosed herein within reference to a gastric band used in restricting the effective size of the stomach for application in bariatric procedures. As such, the implantable restriction device of the present invention is referred to as a gastric band throughout the present disclosure, although those skilled in the art will appreciate the concepts underlying the present invention may be applied in a variety of implantable restriction devices.

The present invention provides for various gastric band constructions addressing the problems associated with hybrid gastric procedures combining surgical gastric reduction with gastric banding. The present invention, therefore allows for the placement of a gastric band 10 directly over a staple line 11 created in the formation of a sleeve portion during the gastric bypass surgery of the hybrid procedure. The gastric band 10 constructions employed in accordance with the present invention protect the gastric band 10 from undesirable interaction with the staples, both during the implantation process and after the gastric band is fully applied about the stomach, in a manner minimizing the potential for puncture of the gastric band 10 by the staples and similar interactions which may cause damage to the staple line 11 due to rubbing of the staple line 11 by positioning of the gastric band 10 thereover. Infections at the placement site of the gastric band 10 are also minimized through utilization of the present gastric band 10.

Although it is contemplated various gastric band constructions may be employed in accordance with the present invention, a preferred gastric band 10 construction is disclosed. More details regarding gastric band construction appropriate for use in conjunction with the present invention may be found in commonly owned U.S. patent application Ser. No. 11/798,634, entitled “Gastric band composed of different hardness materials”, which is incorporated herein by reference.

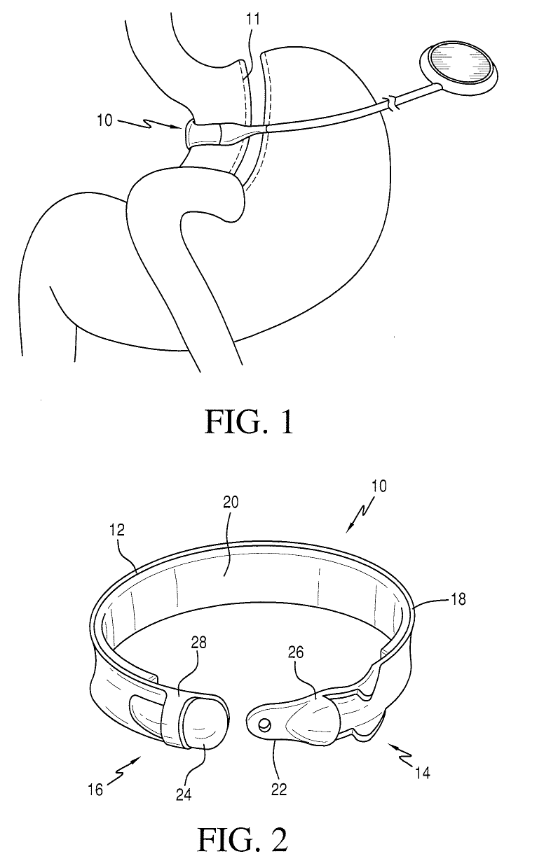

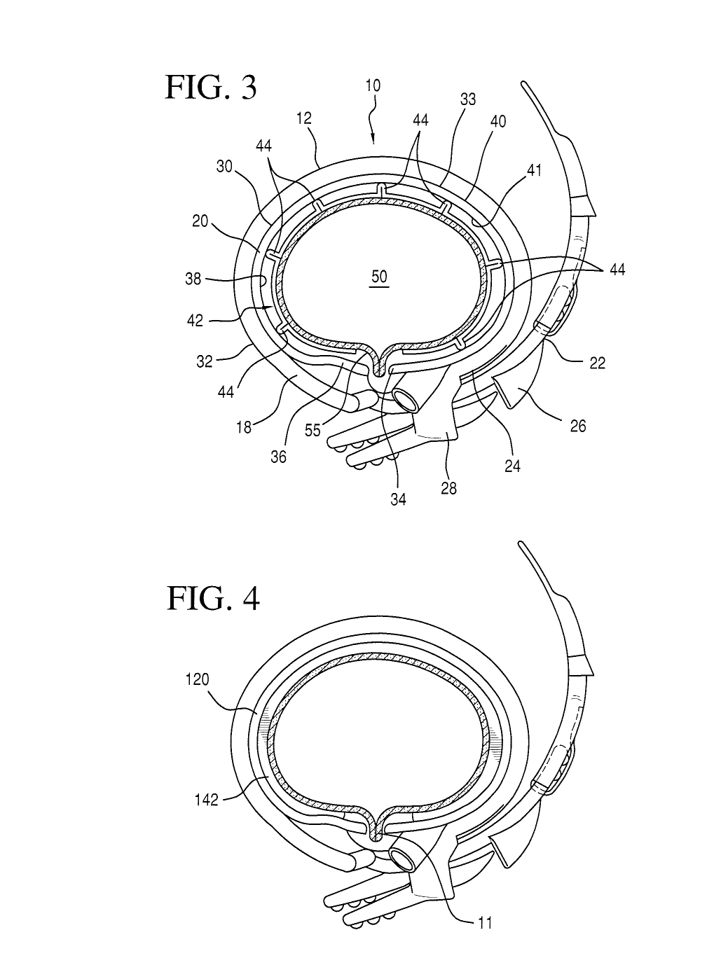

In general, and with reference to FIGS. 1, 2 and 3, the gastric band 10 includes a band body 12 having a first end 14 and an opposite second end 16. The band body 12 and latching mechanism are preferably manufactured from silicone. In accordance with a preferred embodiment of the present invention, the gastric band 10 is a balloon-type gastric band. With this in mind, the gastric band 10 is generally composed of a reinforcing belt 18 to which an elongated balloon 20 is secured. The belt 18 includes a first end 22 and a second end 24 to which the first and second latching members 26, 28 are respectively secured. The belt 18 further includes an inner surface 30 and an outer surface 32 as best shown in FIG. 3. The outer surface 32 is substantially smooth and forms a substantial portion of the outer surface of the gastric band 10 when it is secured about a patient's stomach. The inner surface 30 of the belt 18 is shaped and dimensioned for attachment to the outer surface 33 of the balloon 20.

With regard to the balloon 20, it also includes a first end 34, a second end 36, an inner surface 38 and an outer surface 40. The inner surface 38 is substantially smooth and is shaped and dimensioned for engaging the patient's stomach when the gastric band 10 is secured thereto. The outer surface 40 of the balloon 20 is shaped and dimensioned for coupling with the inner surface 30 of the belt 18.

The gastric band 10 is shaped and dimensioned to circumscribe the stomach at a predetermined location providing for a controlled reduction in the size of the stomach. The gastric band 10 employs a flexible latching mechanism, composed of latching members 26, 28, capable of locking and unlocking without destruction of the latching mechanism or significant reduction in retention capabilities after re-locking. The first and second ends 22, 24 of the belt 18 respectively act as both male and female members depending on the direction of motion and intent to lock or unlock the latching mechanism of the present gastric band 10.

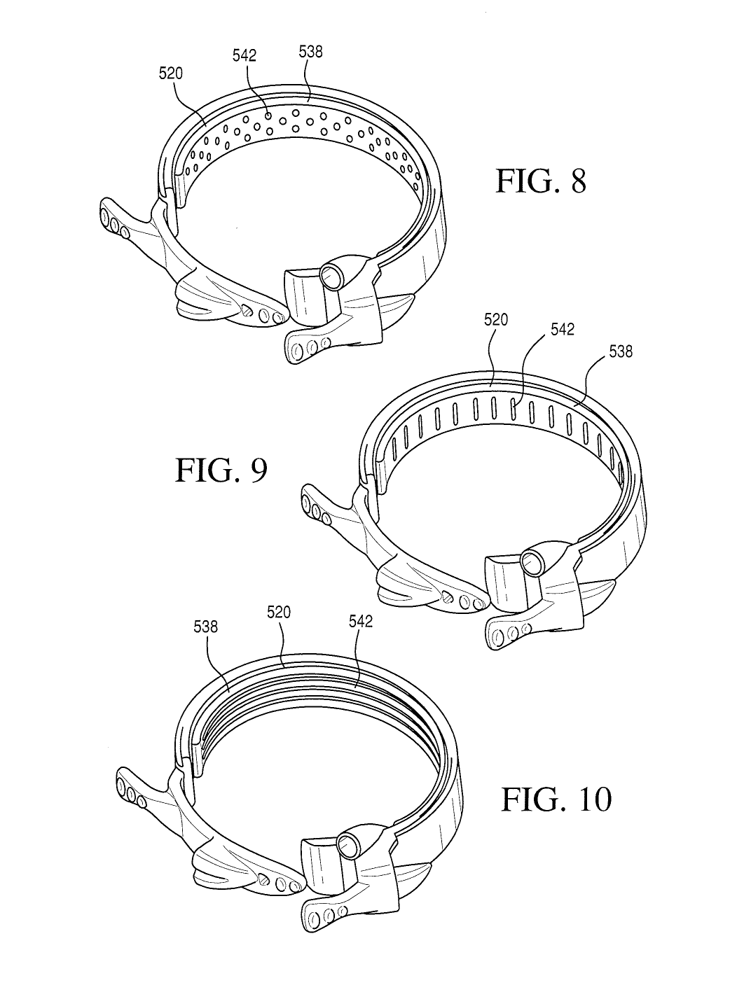

The first end 22 includes a shell member which is generally M-shaped as best shown in FIGS. 8-10, or first latching member 26, generally composed of a hollow, half-moon shaped shell with a tab for gripping and pulling through a collar member, or second latching member 28, composed of a semi-circular shaped aperture on the second end 24. The half-moon shell of the first latching member 26 collapses as it is pulled or pushed through the collar member of the second latching member 28 by a grasper. The collar member includes a tongue such that the shell member slides through the semi-circular shaped aperture and under the tongue during latching. Once the shell member passes the tongue, the roles change. The first end functions as a female component when the shell member resiliently returns to its original shape and is allowed to slide back onto the second end (now a male component) and over the tongue. As such, the shell member functions as both a male component and female component during operation of the latching mechanism and the collar member functions as both a male component and female component during operation of the latching mechanism; that is, the shell member functions as a male component during insertion through the collar member and a female component thereafter when the tongue is seated therein. Unlocking is achieved by employing graspers to pull the first end forward away from the second end removing the tongue from the shell member. The M-shape of the shell member permits it to collapse and move under the tongue and through the collar member.

More particularly, the shell member at the first end of the gastric band 10 is generally a half-moon shaped shell with an open, wide end tapering toward a narrow end adjacent the tip of the first end. The shell member is substantially hollow and is formed from a material, for example, silicone, which permits compression and expansion thereof.

Further details of the operation of the latching mechanism can be found in commonly owned U.S. patent application Ser. No. 11/182,072, entitled “LATCHING DEVICE FOR GASTRIC BAND”, which is incorporated herein by reference.

As discussed above, gastric bands are utilized in conjunction with surgical procedures formed along the stomach. As such, interaction between the interior surface 41 of the gastric band 10 and the staple line created as a result of a surgical procedure are at times encountered. As such, concerns are created regarding this interation and the present gastric band 10 includes mechanisms developed to minimize potential damage to gastric bands when applied over a staple line. These mechanisms relate specifically to the interior surface 38 of the balloon 20 for improving the interaction between the balloon 20 and the staple line.

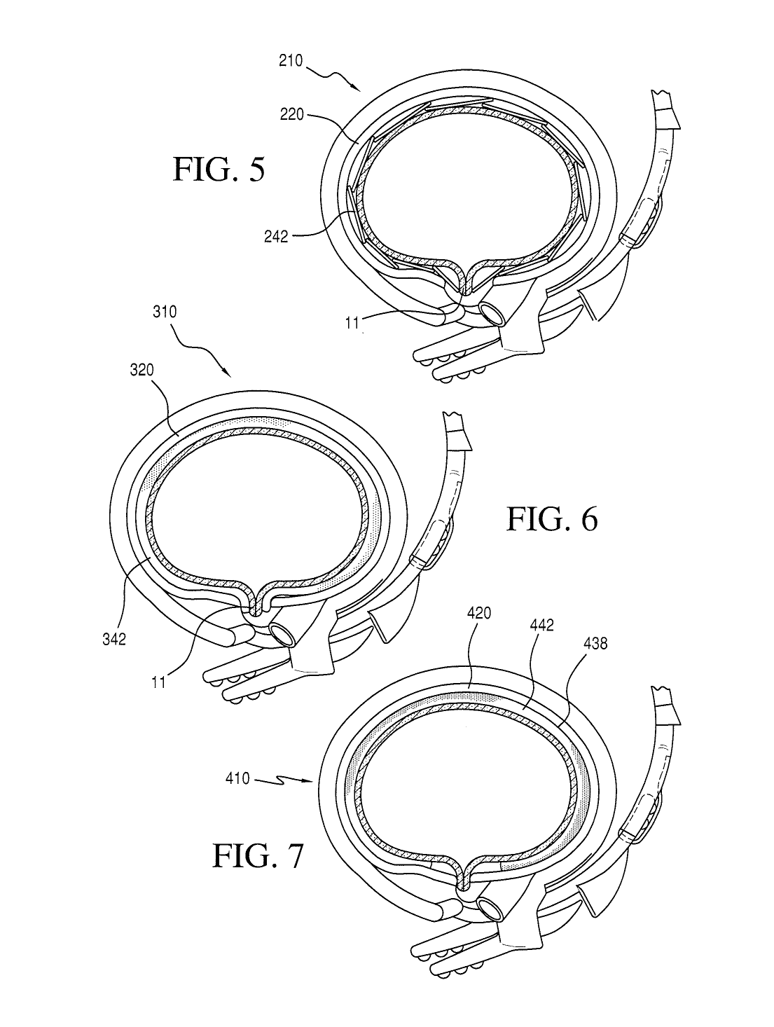

With reference to FIGS. 3, 4 and 5, and in accordance with a preferred embodiment, folded, compressible and unidirectional elastic plates are utilized to cover the surgical staple line to prevent damage to the inflatable portion of the band. The protective plates disclosed in these embodiments may be integrally formed with the gastric band.

Referring now to the embodiment shown with reference to FIG. 3, the protective plate 42 is in the form of a flexible or foldable or pleated plastic sheet which is positioned along the inner surface 38 of the balloon 20 of the gastric band 10. The plate 42 is oriented to space the balloon 20 from the staple line so as to prevent undesirable interaction which might ultimately cause damage to either the staple line or the balloon 20 or both. Spacing is achieved by creating radially extending pleat members 44 along the plate 42 which extend outwardly and act to move the inner surface 38 of the balloon 20 away from the exterior surface 55 of the stomach 50.

Referring to the embodiment shown in FIG. 4, the protective plate 142 is in the form of a directional flexible plate, that is, a plate which may selectively expand or contract along a single longitudinal axis. In accordance with a preferred embodiment, the plate 142 is made of expandable material such as PTFE or a composite fiber matrix elastomer. While these materials are contemplated for use in accordance with a preferred embodiment, it is contemplated other materials capable of expanding/contracting along one plane without changing the size in the other planes may be used within the spirit of the present invention. The plate 142 is oriented to space the balloon 120 from the staple line 11 so as to prevent undesirable interaction which might ultimately cause damage to either the staple line or the balloon 120 or both.

Similarly, and with reference to 5, an overlapping plate system 242 may be employed to protect the band 210. The overlapping plate system 242 utilizes an intralock and overlapping construction that can accordion open or close to protect the balloon 220 of the band 210. As with the prior embodiments, the plate system 242 is oriented to space the balloon 220 from the staple line 11 so as to prevent undesirable interaction which might ultimately cause damage to either the staple line or the balloon 220 or both.

Another embodiment is disclosed with reference to FIG. 6. This embodiment employs a sponge based plate 342 oriented to space the balloon 320 from the staple line 11 so as to prevent undesirable interaction which might ultimately cause damage to either the staple line 11 or the balloon 320 or both. The sponge base plate 342 is made from a sponge elastomer or polymer collapsible or compressible matrix that conforms to the band stomach interface to protect the balloon 320 of the gastric band 310 and the surface of the stomach.

In accordance with yet a further embodiment, and with reference to FIG. 7, a composite matrix elastomer or expandable PTFE protective layer 442 is applied to the interior surface 438 of the balloon 420 of the gastric band 410. The protective layer 442 is an expandable material and allows for expansion and contraction of the gastric band as it is deployed and subsequently employed in the restriction of the gastric cavity. In accordance with preferred embodiments, the material is an expandable PTFE which is highly conformable and when deformed shows virtually no elastic strains. The expandable PTFE also provides for unidirectional expansion/contraction.

In accordance with yet a further embodiment and with reference to FIGS. 8, 9 and 10, a fluid filled balloon 520 is placed in contact with a staple line providing a relatively soft interface to the tissue. However, the staple line can be very abrasive thereby damaging the balloon 520 and resulting in fluid leakage. This problem is solved by integrally forming a small, more durable mechanical feature 542 with the balloon 520 allowing for a more robust material to interface with the staple line and/or providing a small gap between the staple line and the interface, that is, the inner surface 538, of the soft balloon 520. In particular, the mechanical feature 542 is in the form of stand offs, such as feet or ribs, overmolded onto the balloon 520. The stand-offs 542 are shaped and dimensioned to straddle the staple line and protect the balloon 520. The stand offs 542 function to shield the balloon 520 from leak causing punctures. As shown in FIG. 8, and in accordance with one embodiment, the feet 542 are essentially small protruding dimples. In accordance with alternate embodiments, the stand offs 542 may be laterally oriented protrusions as shown with reference to FIG. 9 or longitudinally oriented protrusions as shown with reference to FIG. 10. The standoffs 542 as disclosed in accordance with this embodiment would be of a different durometer (higher or lower) and could have increased wall thickness. These features may be overmolded onto the balloon 520 so that no extra connection points or attachments would be needed. Utilization of such stand offs 542 provides for higher band reliability, the feet or ribs provide a stronger material and/or a separation of the staple line from the fluid filled balloon 520; reduce the potential for reduction of slippage and maintain effective restriction of current bands.

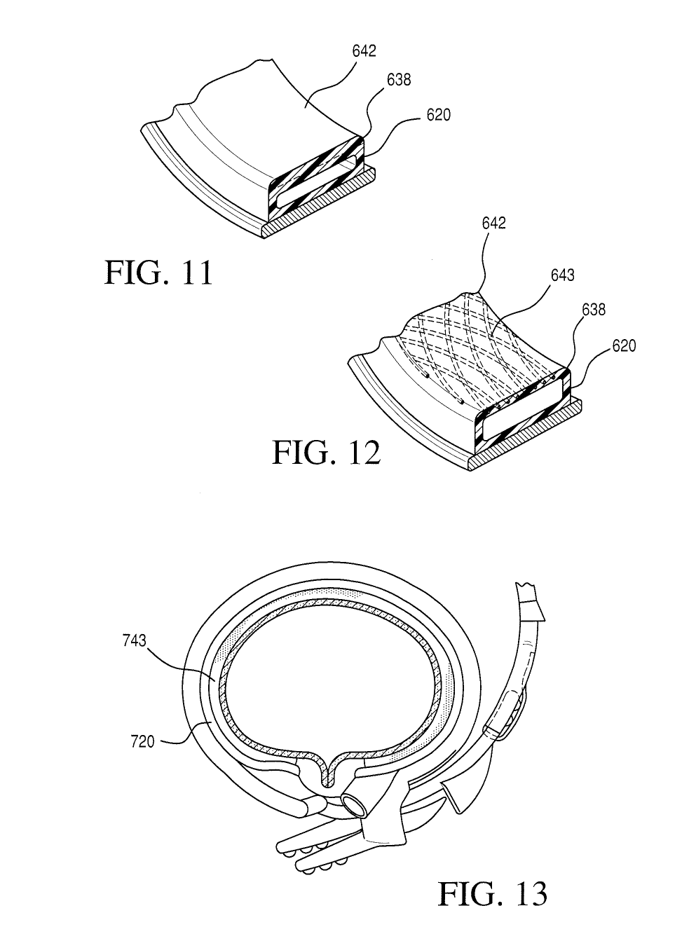

In accordance with yet a further embodiment and referring to FIGS. 11 and 12, an integrally formed protective layer 642 is added to the inner surface 638 of the fluid filled balloon 620 that interfaces with the staple line of the gastric pouch so as to shield the balloon 620 from leak causing punctures. In order to create the protective layer 642, the section of the balloon 620 that is in contact with the staple line, or the entire balloon 620, is formed from a material having a different durometer (higher or lower) material and may also be formed with different wall thicknesses. The balloon 620 of the gastric band would then be capable of withstanding staple penetration without full puncture thereof.

More specifically, the inner surface 638 of the balloon 620 is provided with an integrally formed protective layer 642. In FIG. 12, and in accordance with a variation of the embodiment disclosed with reference to FIG. 11, the protective layer 642 of the balloon 620 contains a fibrous mesh 643 along the inner surface (or wall) 638 of the balloon 620. This allows for a soft interface of the balloon 620 to the stomach but increases the toughness of the balloon wall to deny staple penetration.

In accordance with an alternate embodiment and as shown with reference to FIG. 13, the balloon 720 may have an auxiliary cover 743, such as, an additional sheet of material, for example, Teflon (PTFE), which is positioned in an orientation between the staple line and the fluid filled balloon 720 for the purpose of deflecting staples with which it may come into contact.

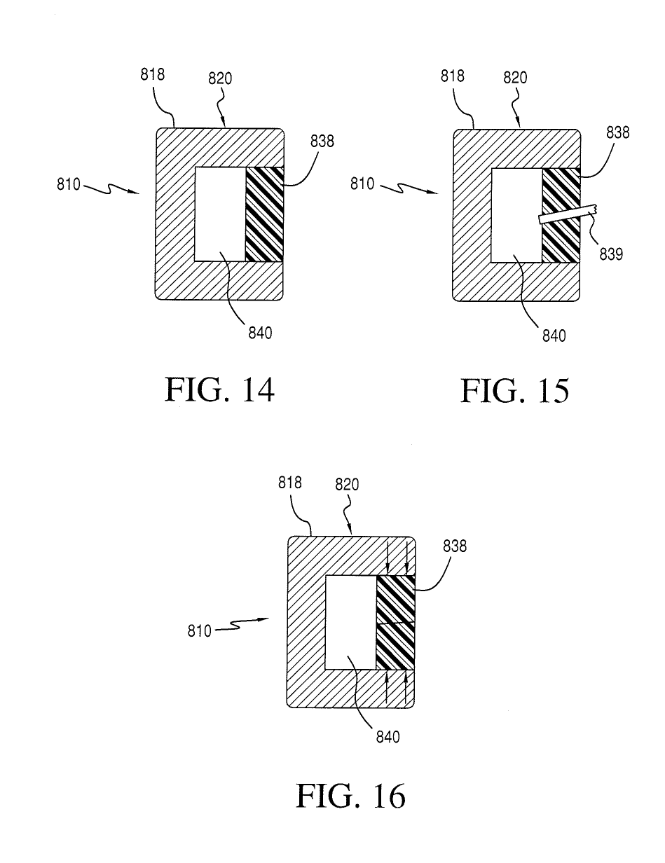

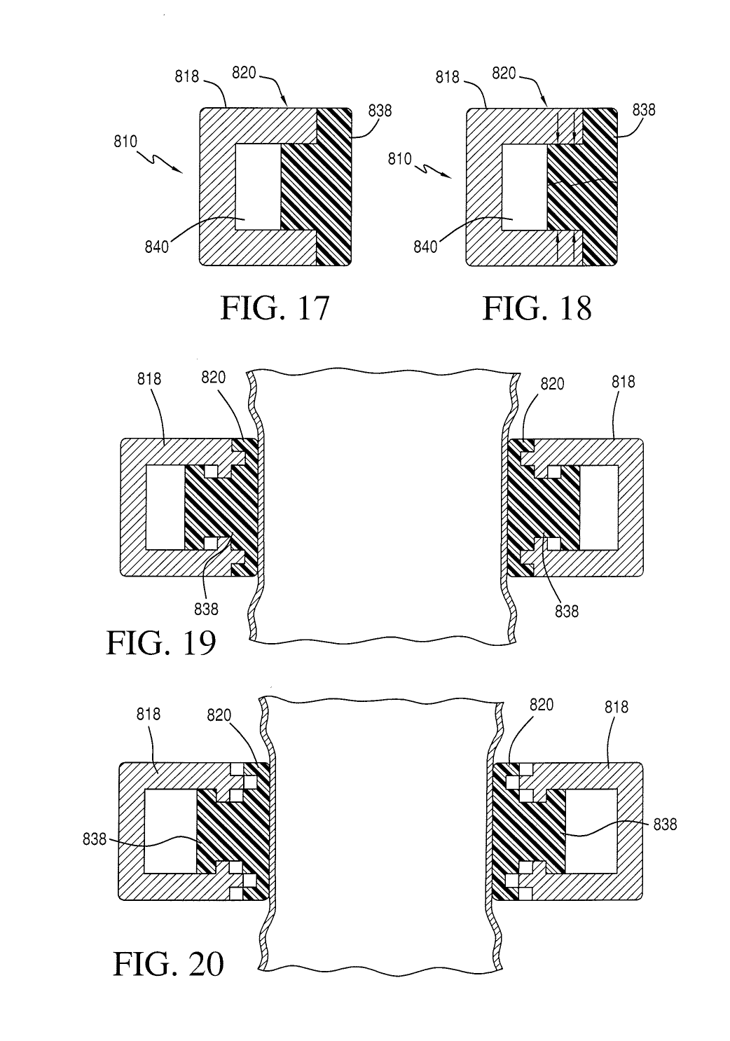

In accordance with yet a further embodiment and with reference to FIG. 14-18, another mechanism for protecting a gastric band 810 is disclosed. As with the prior embodiments, in order to allow a gastric band 810 to be placed over a staple line, the gastric band 810 needs must be more robust than those gastric bands used during conventional gastric banding procedures. Two key obstacles need to be overcome: 1) the gastric band 810 must withstand the potential for puncture caused by exposed staple legs and 2) the long term reliability of the gastric band 810 must not be compromised as a result of having a rigid staple line against the surface of balloon 820. Both of these obstacles are addressed by the present embodiment, which employs a self-healing gastric band 810.

In accordance with this embodiment, the balloon portion 820, that is, the expandable portion of the gastric band 810 that contacts the stomach during application, of the gastric band 810 includes an inner wall 838 that is constructed to self-seal upon puncture thereof. In accordance with such self-sealing properties, the material being punctured is resilient and maintained under compression such that any punctures are immediately closed as a result of the compression (see arrows in FIG. 16) applied to the inner wall 838 during application of the gastric band 810 about the stomach.

In accordance with such an embodiment as disclosed with reference to FIGS. 14, 15 and 16, the inner wall 838 is glued onto a rigid band backbone 818 defining a cavity 840, wherein the inner wall 838, the cavity 840 and the backbone 818 ultimately define the entirety of the balloon 820. This embodiment entails using the same geometry that constrains the silicone septum in a port, allowing puncture by a needle to the inner wall 838 of the balloon 820. FIGS. 14, 15, 16, 17 and 18 provide examples of how such compression could be generated in accordance with the present invention so as to create a closure force causing the inner wall 838 of the balloon 820 of the gastric band 810 to self seal once it is punctured. The pressure from the band material allows the inner wall 838 to be punctured by a staple 839 (see FIG. 15) and still not leak upon removal of the staple 839 (see FIG. 16). Similarly, the pressure from the inner wall 838 will seal about the staple 839 (should the staple not be removed) preventing any leakage from the cavity 840 of the balloon 820. In addition, to get the benefits from compressing the material, the inner wall 838 material would have to be much thicker, reducing the wear potential of the inner wall 838 of the balloon 820 over the staple lines. Such a design would likely require a high-pressure balloon 820. However, and with reference to FIGS. 19 and 20, it is contemplated that a low pressure balloon 820 may be implemented by maintaining the silicone compression and thickness advantage outlined above, but allowing for the inner wall 838 of the balloon 820 to slide out (see FIG. 19 where the inner wall 838 is shown in its nonexpanded position relative to the stomach 50 and FIG. 20 where the inner wall 838 is shown in its expanded position relative to the stomach 50) relative to the backbone 818.

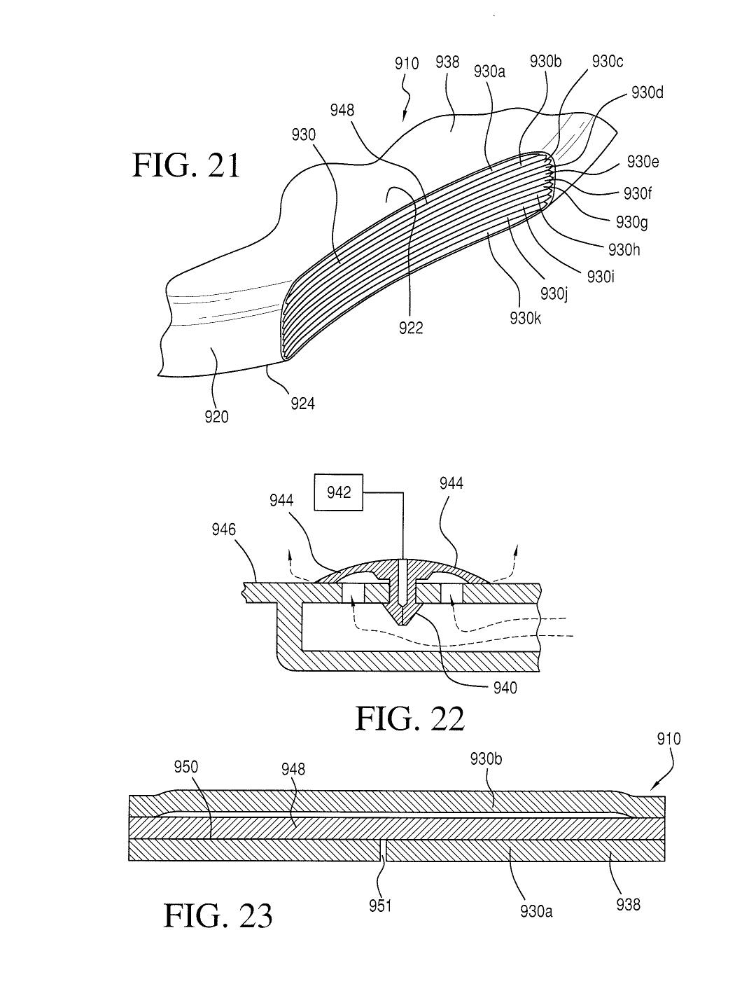

In accordance with an alternate embodiment, the balloon 920 of the gastric band 910 is constructed as a self-healing balloon 920 capable of maintaining positive pressure on the gastric restriction even after puncturing of the balloon 920 from a foreign body. Referring to FIG. 21, the balloon 920 includes a plurality of individual lumens 930 defining radially oriented layers 930a-k extending from the inner most wall of the balloon 920 in towards the belt engaging surface of the balloon 920 that are simultaneously filled via a conventional filling methodology. As such, even when one layer is punctured the lower layers are maintained in an inflated state.

In accordance with a preferred embodiment, the various lumens 930a-k of the balloon 920 are oriented in a layered configuration extending from the inner surface 922 of the balloon 920 toward the outer surface 924 of the balloon 920. Each of the lumens 930a-k is in fluid communication with a fluid source maintaining the lumens 930a-k under pressure sufficient to maintain the balloon 920 in an expanded configuration. Referring to FIG. 23, in the event of puncturing, the reduced pressure in the punctured adjacent lumen 930a would allow for further filling of the adjacent lumen 930b and would compress the punctured lumens 930a, ultimately taking over some of the space previously occupied by the lacerated lumen 930a, thereby maintaining a reduced restriction of the gastric tissue.

While a preferred layering construction is disclosed in accordance with a preferred embodiment of the present invention, it is contemplated a variety of lumen configurations may be employed without departing from the spirit of the present invention. For example, it is contemplated each layer of the balloon may be broken up into various segments. This would allow for even greater control of the internal pressure applied by the balloon in the event one of the lumens is lacerated.

With respect to the filling of the various lumens 930a-k, it is contemplated filling may be controlled by an inlet valve (for example, a duckbill valve) 940 allowing controlled fluid flow from the fluid source 942 to the various lumens 930a-k in only one direction. In the event too much pressure is applied to an individual lumen 930a-k, fluid outflow is permitted via a pair of overflow valves (for example, umbrella valves) 944 integrally formed with the inlet valve 940. The overflow valves 944 have a cracking pressure greater than the maximum inflation pressure of the band 910. Referring to FIG. 22, and in accordance with a preferred embodiment of the present invention, the inlet valve 942 and the overflow valves 944 are formed in a single piece valve assembly. This inlet valve 942 and overflow valves 944 are positioned along the wall 946 of the lumens (for example, lumen 930a as shown in FIG. 22) and allow for controlled inflation thereof without the need for worrying that the lumen 930a will be inflated beyond its predetermined maximum pressure.

As briefly discussed above, FIG. 23 shows the self-healing phenomena of the multilumen band 910 wherein pressure from the inflated lumen 930b occludes the puncture 951 formed in the lumen 930a. More particularly, and as discussed above, the adjacent lumen 930b, when it is expanded, takes over the space of the lacerated lumen 930a formed along the inner wall 938 of the balloon. As a result of this fact, the wall 948 positioned between adjacent lumens 930a and 930b will be forced into an overlying relationship with the inner surface 950 of the lacerated inner wall 938 of the first lumen 930a. The overlying relationship ultimately results in a covering of the puncture, which seals off the lacerated lumen 930a from further negative effects resulting from the laceration.

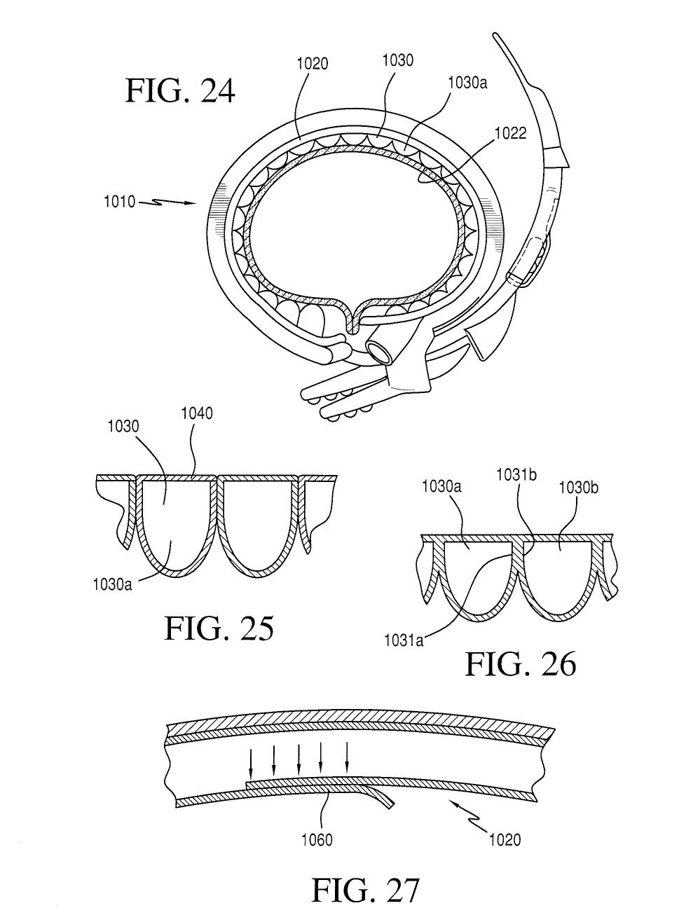

Referring now to FIG. 24, an alternate embodiment is disclosed. This embodiment employs a microcell structure 1030 along the inner wall 1022 of the balloon 1020. As a result of the microcell structure 1030, laceration of a single cell 1030a does not have an effect upon the other cells of the inner wall 1022 and the negatives effects of the laceration are minimized.

The various cells 1030a making up the microcell wall 1030 are composed of independent linked inflatable cells 1030a which minimize damage to the overall gastric band 1010 if one of the cells 1030a happens to be punctured. In accordance with various cell structures and with reference to FIG. 25, chamber isolating flaps 1040 may be positioned between the primary cells 1030a. The chamber isolating flaps 1040 allow for each cell 1030a to act separately if an adjacent cell 1030a is damaged. Referring to FIG. 26, the cells 1030a, 1030b are to be constructed with adjacent walls 1031a, 1031b directly bonded together. The balloon could also be constructed with an internal coating that seals the microcell structure of the balloon in the event of a puncture. The coating may take a variety of forms and compositions known to those skilled in the art.

In accordance with another embodiment and with reference to FIG. 27, the balloon 1020 may be provided with an inflation inlet flap 1060 which self seals upon the application of the pressure to the balloon 1020.

Although the present invention is described for use in conjunction with gastric bands, those skilled in the art will appreciate the above invention has equal applicability to other types of implantable bands, for example, bands that are used for the treatment of fecal incontinence. One such band is described in U.S. Pat. No. 6,461,292. Bands can also be used to treat urinary incontinence. One such band is described in U.S. Patent Application Publication No. 2003/0105385. Bands can also be used to treat heartburn and/or acid reflux. One such band is described in U.S. Pat. No. 6,470,892. Bands can also be used to treat impotence. One such band is described in U.S. Patent Application Publication No. 2003/0114729.

While the preferred embodiments have been shown and described, it will be understood that there is no intent to limit the invention by such disclosure, but rather, is intended to cover all modifications and alternate constructions falling within the spirit and scope of the invention.

1. An implantable restriction device, comprising:
a belt;
a balloon secured to the belt for engagement with tissue when the implantable restriction device is positioned about an organ;
a protective member associated with the balloon for positioning between the balloon and a tissue surface defining a band tissue interface.

2. The implantable restriction device according to claim 1, wherein the protective member includes a protective plate which is flexible and positioned along an inner surface of the balloon of the restrictive device.

3. The implantable restriction device according to claim 2, wherein the protective member includes a protective plate which is pleated and positioned along the inner surface of the balloon of the restrictive device.

4. The implantable restriction device according to claim 2, wherein the protective plate is a directional flexible plate.

5. The implantable restriction device according to claim 2, wherein the protective plate is made of expandable PTFE.

6. The implantable restriction device according to claim 1, wherein the protective member is an overlapping plate system and the overlapping plate system includes an intralocking and overlapping construction that can accordion open or close to protect the balloon.

7. The implantable restriction device according to claim 1, wherein the protective member is a sponge that conforms to the band tissue interface so as to prevent undesirable interaction which might ultimately cause damage to either the staple line or the balloon.

8. The implantable restriction device according to claim 1, wherein the protective member is an expandable PTFE applied to the inner surface of the balloon of the implantable restriction device.

9. The implantable restriction device according to claim 1, wherein the protective member is composed of stand-offs formed along the inner surface of the balloon.

10. The implantable restriction device according to claim 9, wherein the stand-offs are of a different durometer than the balloon.

11. The implantable restriction device according to claim 9, wherein the stand-offs are shaped and dimensioned to straddle the staple line and protect the balloon.

12. The implantable restriction device according to claim 1, wherein the protective member is a protective layer added to the balloon that interfaces with the staple line and wherein a section of the balloon in contact with the staple line is formed from a material having a different durometer than the remainder of the balloon.

13. The implantable restriction device according to claim 1, wherein the protective member is a fibrous mesh within an inner wall of the balloon.

14. The implantable restriction device according to claim 1, wherein the protective member is a self sealing material.

15. The implantable restriction device according to claim 14, wherein the self sealing material is an inner wall of the balloon.

16. The implantable device according to claim 1, wherein the protective member is integrally associated with the balloon.

17. The implantable restriction device according to claim 1, wherein the protective member includes a plurality of individual lumens in a layered configuration.
